(19) United States
(12) Patent Application Publication
(10) Pub. No.: US 2024/0358589 A1
(43) Pub. Date: Oct. 31, 2024

(54) EXTERNAL COUNTER PULSATION DEVICE WITH IMPROVED TIMING AND CONTROL

(71) Applicant: CoCardio, Inc., Encinitas, CA (US)

(72) Inventor: Pratiksha Girish GANDHI, Encinitas, CA (US)

(21) Appl. No.: 18/649,898

(22) Filed: Apr. 29, 2024

Related U.S. Application Data
(60) Provisional application No. 63/498,871, filed on Apr. 28, 2023.

(51) Int. Cl. A61H 31/00

(52) U.S. Cl. CPC A61H 31/005; A61H 2230/045; A61M 2205/3553

(57) ABSTRACT

A system comprising: at least one pressure device configured to inflate and deflate; at least one hardware processor; and software that is configured to, when executed by the at least one hardware processor, automatically initiate inflation and deflation of the at least one pressure devices in synchrony with a subject's cardiac cycle, receive ECG and pressure data, analyze the ECG and pressure data and determine timing and pressure required to achieve inflation and deflation of the at least one pressure device in synchrony with the subject's cardiac cycle, and automatically adjust at least one of timing and pressure of the inflation and deflation of the at least one pressure device to achieve synchrony with the subject's cardiac cycle.

### CROSS-REFERENCE TO RELATED APPLICATIONS

This application claims priority to U.S. Provisional Patent App. No. 63/498,871, entitled “AI Empowered External Counter Pulsation Medical Device With Remote Monitoring Telehealth Capability and EMR Integration,” filed on Apr. 28, 2023, which is incorporated herein by reference as if set forth in full.

### BACKGROUND

### Field of the Invention

The embodiments described herein are generally directed to an external counter pulsation device, and more particularly, to systems and methods for remote monitoring and improved timing control.

### Description of the Related Art

External counter pulsation widely known as ECP is a cardiac medical device used for treating chronic resistant angina cases by cardiologists, physicians in private clinics and cardiology departments in Hospitals. Conventionally, an ECP device is operated by medical technicians daily for one hour for 35 sittings. The law requires a physician to be on-site, but not for direct supervision of the patients. The adoption of this beneficial technology has been very low as the current devices are not capable of allowing the physician to view or intervene in the treatment process or monitor its accuracy. Also, the operator training requires a lot of time and expertise to understand the electrocardiogram (ECG) waveforms, the timing of related to the ECP treatment, and the perfect waveforms that must be generated for the best results, as explained in the Detailed Description. There is also is no integration with Electronic Medical Record (EMR), which makes the work of the medical and billing team cumbersome as every sitting of each 35 sittings must be billed separately. There are 12 million chronic resistant angina patients with significant chest pain in the US alone, and less than a million are currently served by ECP as a result of these issues.

ECP therapy is approved by the Food and Drug Administration (FDA) for treating chronic stable angina that doesn't respond to other treatments. It may also be recommended for individuals who are not eligible for heart surgery. The goal is to reduce the need for medication and improve the ability to be active without experiencing symptoms.

ECP treatment involves applying pressure to blood vessels in the lower limbs. This increased pressure helps improve blood flow back to the heart, allowing the heart to work more efficiently. ECP therapy can also encourage the development of “natural bypass” vessels, which help relieve angina symptoms when coronary arteries are narrowed or blocked.

ECP can be used to treat chronic stable angina (chest pain or pressure), cardiac syndrome X (a type of angina), cerebrovascular disease, heart failure, kidney (renal) failure, left ventricular dysfunction (early stage of heart failure), lung disease (pulmonary disease), and peripheral artery (vascular) disease (PAD). But ECP cannot treat unstable angina (acute coronary syndrome), which presents more severe, frequent, and longer-lasting symptoms.

Angina, also known as angina pectoris, is chest pain or pressure that typically occurs when the heart muscle (myocardium) does not receive sufficient oxygen-rich blood. It is most commonly a symptom of coronary artery disease. Symptoms of Angina include chest pain or a sensation of weight or crushing in the chest, pain in the jaw, arms, neck, throat, stomach area, shoulder, or back often accompanying chest tightness. Other symptoms include nausea, exhaustion, shortness of breath, unexplained sweating, dizziness, light-headedness, weakness, vomiting, and restlessness. Angina occurs when there is restricted blood supply to the heart muscles and is a symptom of a heart condition, not a disease. Types of angina include stable angina where chest pain follows a regular pattern, occurring after physical or mental exertion. Unstable angina is a condition where the symptoms worsen even at rest and may be a medical emergency. Variant angina (Prinzmetal's angina) is a rare form caused by coronary artery spasms at rest.

Risk factors for angina include hypertension, high-fat diet, lack of physical activity, tobacco use, diabetes, family history, advanced age, emotional anxiety, obesity, and stress. And angina can lead to heart attack, sudden cardiac death, and fainting

### SUMMARY

Accordingly, systems, methods, and non-transitory computer-readable media are disclosed to systems and methods for remote monitoring and improved timing control.

According to one aspect, a system comprising: at least one pressure device configured to inflate and deflate; at least one hardware processor; and software that is configured to, when executed by the at least one hardware processor, automatically initiate inflation and deflation of the at least one pressure devices in synchrony with a subject's cardiac cycle, receive ECG and pressure data, analyze the ECG and pressure data and determine timing and pressure required to achieve inflation and deflation of the at least one pressure device in synchrony with the subject's cardiac cycle, and automatically adjust at least one of timing and pressure of the inflation and deflation of the at least one pressure device to achieve synchrony with the subject's cardiac cycle.

It should be understood that any of the features in the methods above may be implemented individually or with any subset of the other features in any combination. Thus, to the extent that the appended claims would suggest particular dependencies between features, disclosed embodiments are not limited to these particular dependencies. Rather, any of the features described herein may be combined with any other feature described herein, or implemented without any one or more other features described herein, in any combination of features whatsoever. In addition, any of the methods, described above and elsewhere herein, may be embodied, individually or in any combination, in executable software modules of a processor-based system, such as a server, and/or in executable instructions stored in a non-transitory computer-readable medium.

### BRIEF DESCRIPTION OF THE DRAWINGS

The details of the present invention, both as to its structure and operation, may be gleaned in part by study of the accompanying drawings, in which like reference numerals refer to like parts, and in which:

FIG. 1 illustrates an example infrastructure, in which one or more of the processes described herein may be implemented, according to an embodiment;

FIG. 2 illustrates an example processing system, by which one or more of the processes described herein may be executed, according to an embodiment;

FIG. 3 illustrates an example ECP device;

FIG. 4 illustrates an example control console assembly that can be included in the ECP device of FIG. 3;

FIG. 5 illustrates an example ECG signal;

FIG. 6 illustrates synchronization of inflation and deflation of the pressure devices included in the device of FIG. 3;

FIG. 7 illustrates graphs of the aortic pressure relative to the ECG;

FIG. 8 illustrates example sequential timing of pressure devices included in the device of FIG. 3;

FIG. 9 illustrates timing of deflation of pressure devices included in the device of FIG. 3;

FIG. 10 illustrates aortic root pressure in the device of FIG. 3; and

FIG. 11 illustrates a process of timing and pressure control, in accordance with on example of embodiment.

### DETAILED DESCRIPTION

In an embodiment, systems, methods, and non-transitory computer-readable media are disclosed for systems and methods for remote monitoring and improved timing control.

After reading this description, it will become apparent to one skilled in the art how to implement the invention in various alternative embodiments and alternative applications. However, although various embodiments of the present invention will be described herein, it is understood that these embodiments are presented by way of example and illustration only, and not limitation. As such, this detailed description of various embodiments should not be construed to limit the scope or breadth of the present invention as set forth in the appended claims.

### 1. System Overview

### 1.1. Infrastructure

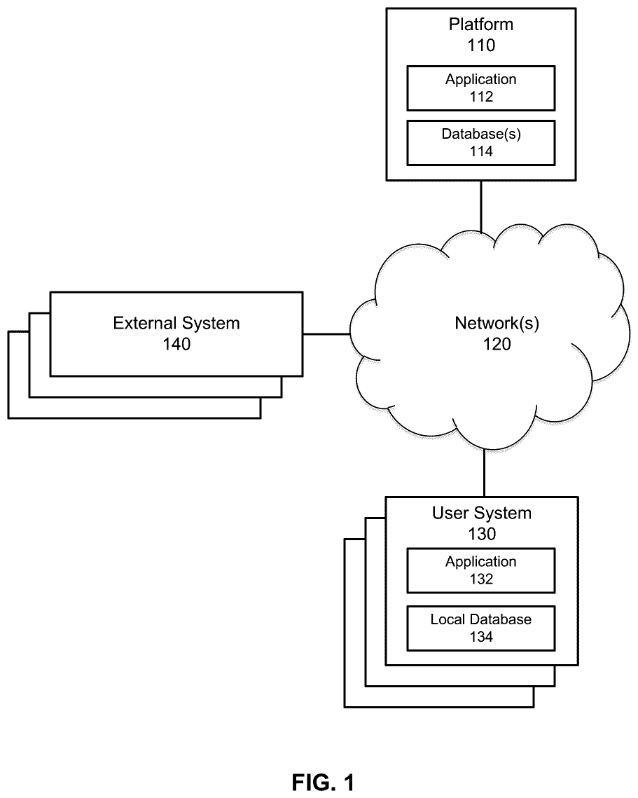

FIG. 1 illustrates an example infrastructure in which one or more of the disclosed processes may be implemented, according to an embodiment. The infrastructure may comprise a platform 110 (e.g., one or more servers) which hosts and/or executes one or more of the various processes (e.g., methods or functions, implemented as software modules) described herein. Platform 110 may comprise dedicated servers, or may instead be implemented in a computing cloud, in which the resources of one or more servers are dynamically and elastically allocated to multiple tenants based on demand. In either case, the servers may be collocated and/or geographically distributed. Platform 110 may also comprise or be communicatively connected to a server application 112 and/or one or more databases 114. In addition, platform 110 may be communicatively connected to one or more user systems 130 via one or more networks 120. Platform 110 may also be communicatively connected to one or more external systems 140 (e.g., other platforms, websites, etc.) via one or more networks 120.

Network(s) 120 may comprise the Internet, and platform 110 may communicate with user system(s) 130 through the Internet using standard transmission protocols, such as HyperText Transfer Protocol (HTTP), HTTP Secure (HTTPS), File Transfer Protocol (FTP), FTP Secure (FTPS), Secure Shell FTP (SFTP), and the like, as well as proprietary protocols. While platform 110 is illustrated as being connected to various systems through a single set of network(s) 120, it should be understood that platform 110 may be connected to the various systems via different sets of one or more networks. For example, platform 110 may be connected to a subset of user systems 130 and/or external systems 140 via the Internet, but may be connected to one or more other user systems 130 and/or external systems 140 via an intranet. Furthermore, while only a few user systems 130 and external systems 140, one server application 112, and one set of database(s) 114 are illustrated, it should be understood that the infrastructure may comprise any number of user systems, external systems, server applications, and databases.

User system(s) 130 may comprise any type or types of computing devices capable of wired and/or wireless communication, including without limitation, desktop computers, laptop computers, tablet computers, smart phones or other mobile phones, servers, game consoles, televisions, set-top boxes, electronic kiosks, point-of-sale terminals, and/or the like. Each user system 130 may comprise or be communicatively connected to a client application 132 and/or one or more local databases 134.

Platform 110 may comprise web servers which host one or more websites and/or web services. In embodiments in which a website is provided, the website may comprise a graphical user interface, including, for example, one or more screens (e.g., webpages) generated in HyperText Markup Language (HTML) or other language. Platform 110 transmits or serves one or more screens of the graphical user interface in response to requests from user system(s) 130. In some embodiments, these screens may be served in the form of a wizard, in which case two or more screens may be served in a sequential manner, and one or more of the sequential screens may depend on an interaction of the user or user system 130 with one or more preceding screens. The requests to platform 110 and the responses from platform 110, including the screens of the graphical user interface, may both be communicated through network(s) 120, which may include the Internet, using standard communication protocols (e.g., HTTP, HTTPS, etc.). These screens (e.g., webpages) may comprise a combination of content and elements, such as text, images, videos, animations, references (e.g., hyperlinks), frames, inputs (e.g., textboxes, text areas, checkboxes, radio buttons, drop-down menus, buttons, forms, etc.), scripts (e.g., JavaScript), and the like, including elements comprising or derived from data stored in one or more databases (e.g., database(s) 114) that are locally and/or remotely accessible to platform 110. It should be understood that platform 110 may also respond to other requests from user system(s) 130.

Platform 110 may comprise, be communicatively coupled with, or otherwise have access to one or more database(s) 114. For example, platform 110 may comprise one or more database servers which manage one or more databases 114. Server application 112 executing on platform 110 and/or client application 132 executing on user system 130 may submit data (e.g., user data, form data, etc.) to be stored in database(s) 114, and/or request access to data stored in database(s) 114. Any suitable database may be utilized, including without limitation MySQL™, Oracle™, IBM™, Microsoft SQL™, Access™, PostgreSQL™, MongoDB™, and the like, including cloud-based databases and proprietary databases. Data may be sent to platform 110, for instance, using the well-known POST request supported by HTTP, via FTP, and/or the like. This data, as well as other requests, may be handled, for example, by server-side web technology, such as a servlet or other software module (e.g., comprised in server application 112), executed by platform 110.

In embodiments in which a web service is provided, platform 110 may receive requests from user system(s) 130 and/or external system(s) 140, and provide responses in extensible Markup Language (XML), JavaScript Object Notation (JSON), and/or any other suitable or desired format. In such embodiments, platform 110 may provide an application programming interface (API) which defines the manner in which user system(s) 130 and/or external system(s) 140 may interact with the web service. Thus, user system(s) 130 and/or external system(s) 140 (which may themselves be servers), can define their own user interfaces, and rely on the web service to implement or otherwise provide the backend processes (e.g., methods and functionality), storage, and/or the like, described herein. For example, in such an embodiment, a client application 132, executing on one or more user system(s) 130, may interact with a server application 112 executing on platform 110 to execute one or more or a portion of one or more of the various process(es) described herein.

Client application 132 may be “thin,” in which case processing is primarily carried out server-side by server application 112 on platform 110. A basic example of a thin client application 132 is a browser application, which simply requests, receives, and renders webpages at user system(s) 130, while server application 112 on platform 110 is responsible for generating the webpages and managing database functions. Alternatively, the client application may be “thick,” in which case processing is primarily carried out client-side by user system(s) 130. It should be understood that client application 132 may perform an amount of processing, relative to server application 112 on platform 110, at any point along this spectrum between “thin” and “thick,” depending on the design goals of the particular implementation. In any case, the software described herein, which may wholly reside on either platform 110 (e.g., in which case server application 112 performs all processing) or user system(s) 130 (e.g., in which case client application 132 performs all processing) or be distributed between platform 110 and user system(s) 130 (e.g., in which case server application 112 and client application 132 both perform processing), can comprise one or more executable software modules comprising instructions that implement one or more of the processes (e.g., methods or functions) described herein.

### 1.2. Example Processing Device

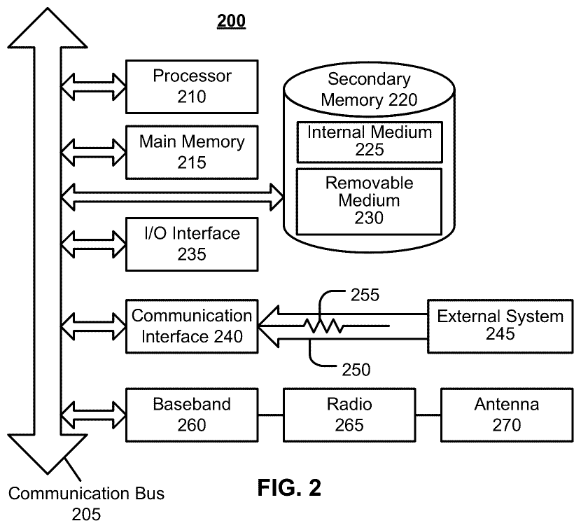

FIG. 2 is a block diagram illustrating an example wired or wireless system 200 that may be used in connection with various embodiments described herein. For example, system 200 may be used as or in conjunction with one or more of the processes (e.g., to store and/or execute the software), including any methods or functions, described herein, and may represent components of platform 110, user system(s) 130, external system(s) 140, and/or other processing devices described herein. System 200 can be any processor-enabled device (e.g., server, personal computer, etc.) that is capable of wired or wireless data communication. Other processing systems and/or architectures may also be used, as will be clear to those skilled in the art.

System 200 may comprise one or more processors 210. Processor(s) 210 may comprise a central processing unit (CPU). Additional processors may be provided, such as a graphics processing unit (GPU), an auxiliary processor to manage input/output, an auxiliary processor to perform floating-point mathematical operations, a special-purpose microprocessor having an architecture suitable for fast execution of signal-processing algorithms (e.g., digital-signal processor), a subordinate processor (e.g., back-end processor), an additional microprocessor or controller for dual or multiple processor systems, and/or a coprocessor. Such auxiliary processors may be discrete processors or may be integrated with a main processor 210. Examples of processors which may be used with system 200 include, without limitation, any of the processors (e.g., Pentium™, Core i7™, Core i9™, Xeon™, etc.) available from Intel Corporation of Santa Clara, California, any of the processors available from Advanced Micro Devices, Incorporated (AMD) of Santa Clara, California, any of the processors (e.g., A series, M series, etc.) available from Apple Inc. of Cupertino, any of the processors (e.g., Exynos™) available from Samsung Electronics Co., Ltd., of Seoul, South Korea, any of the processors available from NXP Semiconductors N.V. of Eindhoven, Netherlands, and/or the like.

Processor(s) 210 may be connected to a communication bus 205. Communication bus 205 may include a data channel for facilitating information transfer between storage and other peripheral components of system 200. Furthermore, communication bus 205 may provide a set of signals used for communication with processor 210, including a data bus, address bus, and/or control bus (not shown). Communication bus 205 may comprise any standard or non-standard bus architecture such as, for example, bus architectures compliant with industry standard architecture (ISA), extended industry standard architecture (EISA), Micro Channel Architecture (MCA), peripheral component interconnect (PCI) local bus, standards promulgated by the Institute of Electrical and Electronics Engineers (IEEE) including IEEE 488 general-purpose interface bus (GPIB), IEEE 696/S-100, and/or the like.

System 200 may comprise main memory 215. Main memory 215 provides storage of instructions and data for programs executing on processor 210, such as any of the software discussed herein. It should be understood that programs stored in the memory and executed by processor 210 may be written and/or compiled according to any suitable language, including without limitation C/C++, Java, JavaScript, Perl, Python, Visual Basic, .NET, and the like. Main memory 215 is typically semiconductor-based memory such as dynamic random access memory (DRAM) and/or static random access memory (SRAM). Other semiconductor-based memory types include, for example, synchronous dynamic random access memory (SDRAM), Rambus dynamic random access memory (RDRAM), ferroelectric random access memory (FRAM), and the like, including read only memory (ROM).

System 200 may comprise secondary memory 220. Secondary memory 220 is a non-transitory computer-readable medium having computer-executable code and/or other data (e.g., any of the software disclosed herein) stored thereon. In this description, the term “computer-readable medium” is used to refer to any non-transitory computer-readable storage media used to provide computer-executable code and/or other data to or within system 200. The computer software stored on secondary memory 220 is read into main memory 215 for execution by processor 210. Secondary memory 220 may include, for example, semiconductor-based memory, such as programmable read-only memory (PROM), erasable programmable read-only memory (EPROM), electrically erasable read-only memory (EEPROM), and flash memory (block-oriented memory similar to EEPROM).

Secondary memory 220 may include an internal medium 225 and/or a removable medium 230. Internal medium 225 and removable medium 230 are read from and/or written to in any well-known manner. Internal medium 225 may comprise one or more hard disk drives, solid state drives, and/or the like. Removable storage medium 230 may be, for example, a magnetic tape drive, a compact disc (CD) drive, a digital versatile disc (DVD) drive, other optical drive, a flash memory drive, and/or the like.

System 200 may comprise an input/output (I/O) interface 235. I/O interface 235 provides an interface between one or more components of system 200 and one or more input and/or output devices. Example input devices include, without limitation, sensors, keyboards, touch screens or other touch-sensitive devices, cameras, biometric sensing devices, computer mice, trackballs, pen-based pointing devices, and/or the like. Examples of output devices include, without limitation, other processing systems, cathode ray tubes (CRTs), plasma displays, light-emitting diode (LED) displays, liquid crystal displays (LCDs), printers, vacuum fluorescent displays (VFDs), surface-conduction electron-emitter displays (SEDs), field emission displays (FEDs), and/or the like. In some cases, an input and output device may be combined, such as in the case of a touch panel display (e.g., in a smartphone, tablet computer, or other mobile device).

System 200 may comprise a communication interface 240. Communication interface 240 allows software to be transferred between system 200 and external devices (e.g. printers), networks, or other information sources. For example, computer-executable code and/or data may be transferred to system 200 from a network server (e.g., platform 110) via communication interface 240. Examples of communication interface 240 include a built-in network adapter, network interface card (NIC), Personal Computer Memory Card International Association (PCMCIA) network card, card bus network adapter, wireless network adapter, Universal Serial Bus (USB) network adapter, modem, a wireless data card, a communications port, an infrared interface, an IEEE 1394 fire-wire, and any other device capable of interfacing system 200 with a network (e.g., network(s) 120) or another computing device. Communication interface 240 preferably implements industry-promulgated protocol standards, such as Ethernet IEEE 802 standards, Fiber Channel, digital subscriber line (DSL), asynchronous digital subscriber line (ADSL), frame relay, asynchronous transfer mode (ATM), integrated digital services network (ISDN), personal communications services (PCS), transmission control protocol/Internet protocol (TCP/IP), serial line Internet protocol/point to point protocol (SLIP/PPP), and so on, but may also implement customized or non-standard interface protocols as well.

Software transferred via communication interface 240 is generally in the form of electrical communication signals 255. These signals 255 may be provided to communication interface 240 via a communication channel 250 between communication interface 240 and an external system 245 (e.g., which may correspond to an external system 140, an external computer-readable medium, and/or the like). In an embodiment, communication channel 250 may be a wired or wireless network (e.g., network(s) 120), or any variety of other communication links. Communication channel 250 carries signals 255 and can be implemented using a variety of wired or wireless communication means including wire or cable, fiber optics, conventional phone line, cellular phone link, wireless data communication link, radio frequency (“RF”) link, or infrared link, just to name a few.

Computer-executable code is stored in main memory 215 and/or secondary memory 220. Computer-executable code can also be received from an external system 245 via communication interface 240 and stored in main memory 215 and/or secondary memory 220. Such computer-executable code, when executed, enable system 200 to perform the various process(es) of the disclosed embodiments as described elsewhere herein.

In an embodiment that is implemented using software, the software may be stored on a computer-readable medium and initially loaded into system 200 by way of removable medium 230, I/O interface 235, or communication interface 240. In such an embodiment, the software is loaded into system 200 in the form of electrical communication signals 255. The software, when executed by processor 210, preferably causes processor 210 to perform one or more of the processes described elsewhere herein.

System 200 may comprise wireless communication components that facilitate wireless communication over a voice network and/or a data network (e.g., in the case of user system 130). The wireless communication components comprise an antenna system 270, a radio system 265, and a baseband system 260. In system 200, radio frequency (RF) signals are transmitted and received over the air by antenna system 270 under the management of radio system 265.

In an embodiment, antenna system 270 may comprise one or more antennae and one or more multiplexors (not shown) that perform a switching function to provide antenna system 270 with transmit and receive signal paths. In the receive path, received RF signals can be coupled from a multiplexor to a low noise amplifier (not shown) that amplifies the received RF signal and sends the amplified signal to radio system 265.

In an alternative embodiment, radio system 265 may comprise one or more radios that are configured to communicate over various frequencies. In an embodiment, radio system 265 may combine a demodulator (not shown) and modulator (not shown) in one integrated circuit (IC). The demodulator and modulator can also be separate components. In the incoming path, the demodulator strips away the RF carrier signal leaving a baseband receive audio signal, which is sent from radio system 265 to baseband system 260.

If the received signal contains audio information, then baseband system 260 decodes the signal and converts it to an analog signal. Then the signal is amplified and sent to a speaker. Baseband system 260 also receives analog audio signals from a microphone. These analog audio signals are converted to digital signals and encoded by baseband system 260. Baseband system 260 also encodes the digital signals for transmission and generates a baseband transmit audio signal that is routed to the modulator portion of radio system 265. The modulator mixes the baseband transmit audio signal with an RF carrier signal, generating an RF transmit signal that is routed to antenna system 270 and may pass through a power amplifier (not shown). The power amplifier amplifies the RF transmit signal and routes it to antenna system 270, where the signal is switched to the antenna port for transmission.

Baseband system 260 is communicatively coupled with processor(s) 210, which have access to memory 215 and 220. Thus, software can be received from baseband processor 260 and stored in main memory 210 or in secondary memory 220, or executed upon receipt. Such software, when executed, can enable system 200 to perform the various process(es) of the disclosed embodiments.

### 1.3. ECP Device

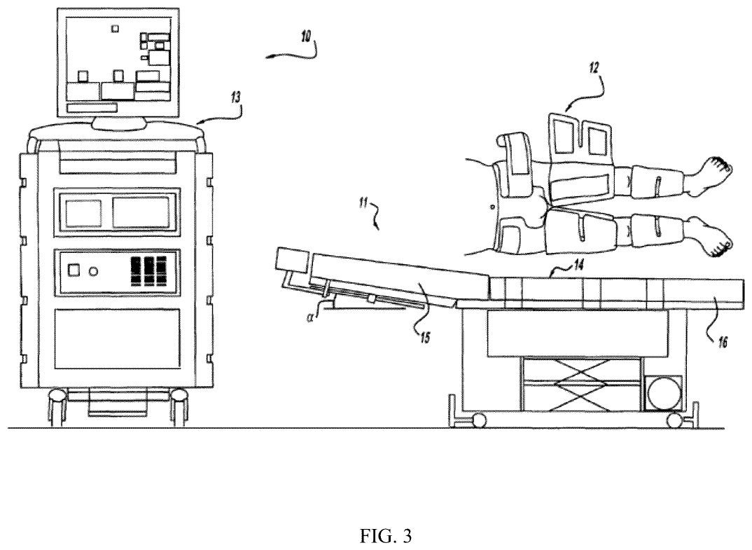

U.S. Pat. No. 7,048,702 (the '702 patent), which is incorporated herein in its entirety as if set forth in describes full, describes a conventional ECP device. FIG. 3 is a recreation of FIG. 1 of the '702 patent. As noted in the '702 Patent, the ECP device 10 described therein and with respect to FIG. 3 (FIG. 1 of the '702 patent) comprises three 3 basic component assemblies, namely: a treatment table assembly 11; a pressure device 12; and control console assembly 13, preferably includ-ing a device for inflating and deflating the pressure devices and a controller that initiates inflation and deflation of the pressure devices. Thus, console assembly 13 can be a user system 132 as described above.

The optimized ECP device 10, as described in the '702 patent, preferably comprises pressure devices 12 that are applied to the legs or other limbs of the subject, preferably to the calves, thighs and buttocks of the subject. Such pressure devices 12 apply pressure to the limb or limbs using, in one embodiment, a bladder that is inflated with a fluid, preferably air. Preferably the pressure device 12 comprises a bladder and a fastener that holds the bladder against the limb or limbs, so that when the bladder is inflated pressure is applied to the limb or limbs. In one embodiment, the fastener comprises a cuff body that holds the bladder against the limb or limbs, preferably a cuff surrounding a bladder.

Preferably, each bladder applies from about one hundred forty (140) to about three hundred twenty (320) millimeters Hg of pressure to the limb. Different sizes of bladders and fasteners may be provided to meet the requirements of different body shapes. Preferably space between the fastener and the bladder and between the bladder and the limb or limbs is minimized.

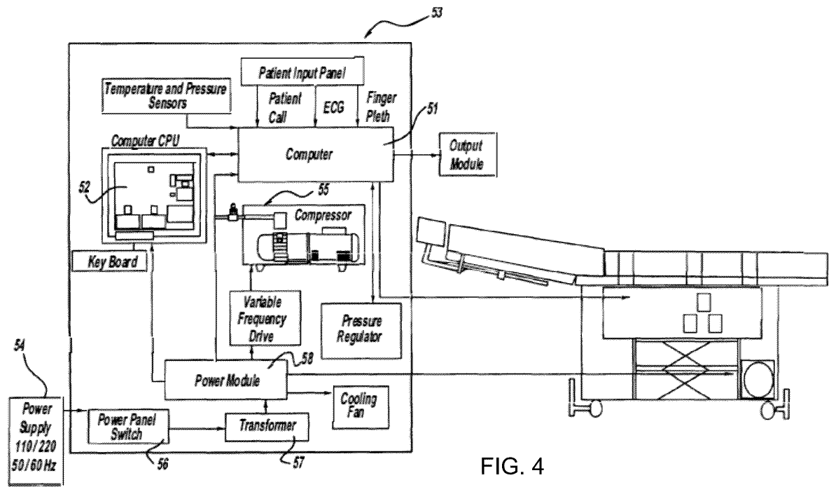

The optimized ECP device 10 also preferably comprises a controller that initiates inflation and deflation of the pressure devices 12 in synchrony with the cardiac cycle of the subject. In one embodiment, the controller is part of a control console assembly. As depicted in FIG. 4, which is a recreation of FIG. 3 in the '702 patent, one control console assembly embodiment generally includes a computer 51, a user interface device 52, such as a computer monitor or touch screen, and a cabinet or housing 53, in which various system components are located and housed. The control console assembly preferably includes a power supply 54 that feeds power to the computer 51 and the compressor assembly 55 by way of a power switch panel 56, transformer 57, and power module 58, which includes a 45 power converter and ramp-up assembly. Preferably, the control console assembly 53 is mounted on wheels for mobility from one location to another upon wheels. Thus, computer 51 can be a processor system 200 as described above.

The user interface 52 is preferably a touch screen monitor for easy monitoring of patient treatment status, treatment parameters, and other relevant data, and provides the capability for adjustment to control operation. In one embodiment, the computer 51 monitors and records information associated with the treatment of the patient. Alternatively, another computer or remote computing system can be used to monitor and record information associated with the treatment of one or more patients. The user interface also provides switches or computer links to switches for adjusting the timing of the inflation/deflation cycle, allowing the operator to adjust the setting of the time for the start of sequential inflation as it is measured relative to the R peak of the treated subject's ECG signal, as further described below.

In one embodiment, the user interface 52 displays treatment information, including an electrocardio-graph (ECG) signal from the subject being treated. As will be apparent to one skilled in the art, the R wave portion of the ECG signal is typically used to monitor the cardiac cycle of the patient. Preferably, the blood flow and/or blood pressure of the patient is also displayed, e.g., to facilitate monitoring of the cardiac cycle of the subject as well as the effect of the counter pulsation waves being applied to the optimized ECP device 10. In one embodiment, the signal is a photo-plethysmograph waveform signal as received from a finger plethysmograph probe.

The controller initiates inflation and deflation of the pressure devices in synchrony with the subject's cardiac cycle. Inflation and deflation are performed so as to create a retrograde pulse of arterial blood that arrives at the heart at approximately the end of the ejection phase of the left ventricle at the time of aortic valve closure. The time of aortic valve closure may be determined through direct or indirect measures. In one embodiment, the time of aortic valve closure can be determined indirectly using finger plethysmography. The synchronization of inflation and deflation of the pressure devices is exemplified in FIG. 4 of the '702 patent.

The safety and effectiveness of the optimized ECP therapy depends on the precise timing of the inflation/deflation cycle in relation to the cardiac cycle of the patient. For example, a hardened arterial wall (i.e., with significant calcium depos-its) will transmit the external pressure pulse up the aorta faster than an elastic artery. Therefore, the inflation valves should be opened later for a calcified artery than for a normally elastic artery. Accordingly, determination of infla-tion and deflation of the pressure devices is preferably adjusted for every individual subject to be treated.

In one embodiment, there are several factors that time of the pressure devices. They include release of all external pressure before the next systole to produce maximal systolic unloading (maximum reduction of systolic pressure), and maintenance of inflation as long as possible to fully utilize the whole period of diastole so as to produce the longest possible diastolic augmentation (maximum increase of diastolic pressure due to externally applied pressure). Therefore, one measurement of effective counter pulsation is the ability to minimize systolic pressure, and at the same time maximize the ratio of the area under the diastolic waveform to that of the area under the systolic waveform.

### 2. Process Overview

Embodiments of processes for systems and methods for remote monitoring and improved timing control will now be described in detail. It should be understood that the described processes may be embodied in one or more software modules that are executed by one or more hardware processors (e.g., processor 210), for example, as a software application (e.g., server application 112, client application 132, and/or a distributed application comprising both server application 112 and client application 132), which may be executed wholly by processor(s) of platform 110, wholly by processor(s) of user system(s) 130, or may be distributed across platform 110 and user system(s) 130, such that some portions or modules of the software application are executed by platform 110 and other portions or modules of the software application are executed by user system(s) 130. The described processes may be implemented as instructions represented in source code, object code, and/or machine code. These instructions may be executed directly by hardware processor(s) 210, or alternatively, may be executed by a virtual machine operating between the object code and hardware processor(s) 210. In addition, the disclosed software may be built upon or interfaced with one or more existing systems.

Alternatively, the described processes may be implemented as a hardware component (e.g., general-purpose processor, integrated circuit (IC), application-specific integrated circuit (ASIC), digital signal processor (DSP), field-programmable gate array (FPGA) or other programmable logic device, discrete gate or transistor logic, etc.), combination of hardware components, or combination of hardware and software components. To clearly illustrate the interchangeability of hardware and software, various illustrative components, blocks, modules, circuits, and steps are described herein generally in terms of their functionality. Whether such functionality is implemented as hardware or software depends upon the particular application and design constraints imposed on the overall system. Skilled persons can implement the described functionality in varying ways for each particular application, but such implementation decisions should not be interpreted as causing a departure from the scope of the invention. In addition, the grouping of functions within a component, block, module, circuit, or step is for case of description. Specific functions or steps can be moved from one component, block, module, circuit, or step to another without departing from the invention.

Furthermore, while the processes, described herein, are illustrated with a certain arrangement and ordering of subprocesses, each process may be implemented with fewer, more, or different subprocesses and a different arrangement and/or ordering of subprocesses. In addition, it should be understood that any subprocess, which does not depend on the completion of another subprocess, may be executed before, after, or in parallel with that other independent subprocess, even if the subprocesses are described or illustrated in a particular order.

### 2.1. Automatic Timing and Pressure Control

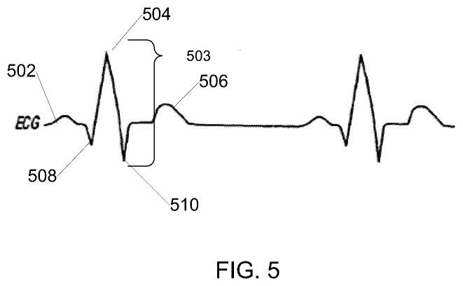

The ECG is important for an effective ECP process as the inflating and deflating of the inflation devices 12 is typically keyed off of the ECG signal, a typical example of which is illustrated in FIG. 5. A normal cardiac cycle as illustrated in FIG. 5, includes a P-wave 502, QRS complex 503, and T-wave 506. When the heart is working properly, all four chambers beat in an organized manner. A cardiac cycle is the result of an electrical impulse that travels through the heart and initiates contraction of its muscle. The P-wave 502 represents atrial contraction or the onset of atrial systole. The QRS complex 503 includes several waveforms, the Q-wave 508, R-wave 504, and S-wave 510. The R-wave 504 is generally the most prominent part of the complex 503. The QRS complex 503 represents the beginning of the systolic phase and of contraction of the ventricles. Arial diastole is also occurring but cannot be seen because ventricular systole has a greater electrical signal. The T-wave 506 represents relaxation of the ventricles or the onset of the diastolic phase.

As noted in the '702 patent, the user interface 52 can display treatment information, including an electrocardio-graph (ECG) signal from the subject being treated. As noted, the R-wave portion 504 of the ECG signal is typically used to monitor the cardiac cycle of the patient. Preferably, the blood flow and/or blood pressure of the patient is also displayed, e.g., to facilitate monitoring of the cardiac cycle of the subject as well as the effect of the counter pulsation waves being applied to the optimized ECP apparatus.

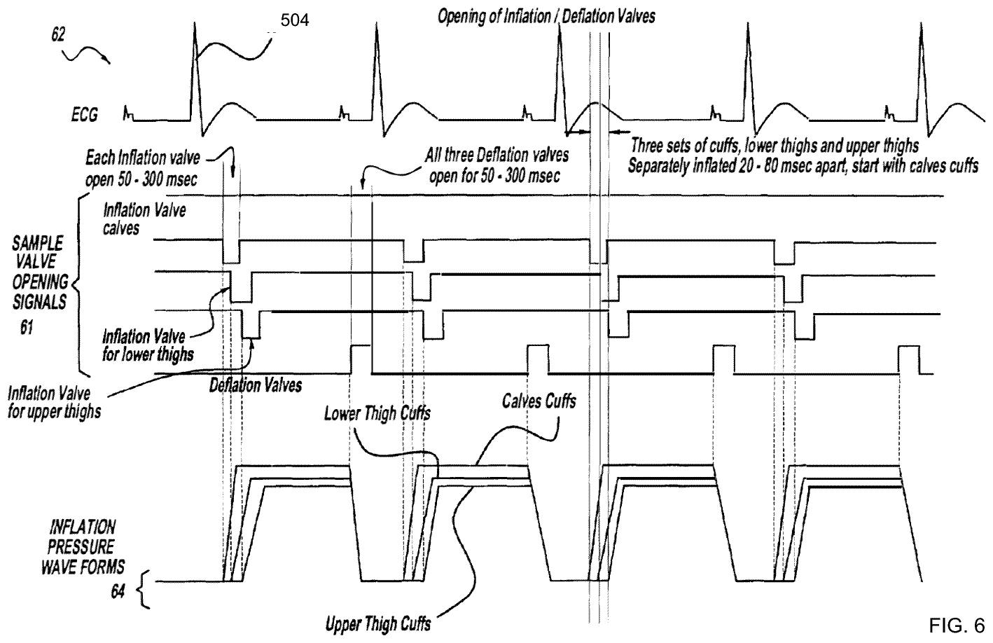

The controller initiates inflation and deflation of the pressure devices in synchrony with the subject's cardiac cycle. Inflation and deflation are affected so as to create a retrograde pulse of arterial blood that arrives at the heart at approximately the end of the ejection phase of the left ventricle at the time of aortic valve closure. The time of aortic valve closure may be determined through direct or indirect measures. In a preferred embodiment, the time of aortic valve closure is determined indirectly using finger plethysmography. The synchronization of inflation and deflation of the pressure devices is exemplified in FIG. 4 of the '702 patent, which is recreated here as FIG. 6. As shown, the controller generates signals 61 for opening and closing valves that release air from the pressure tank to the pressure devices 12. These signals are synchronized with the treated subject's ECG 62, with the R-wave 504 as a trigger point. The pressure devices are inflated sequentially as shown by pressure waveforms 64.

The safety and effectiveness of the optimized ECP therapy depends on the precise timing of the inflation/deflation cycle in relation to the cardiac cycle of the patient. For example, a hardened arterial wall (i.e., with significant calcium deposits) will transmit the external pressure pulse up the aorta faster than an elastic artery. Therefore, the inflation valves should be opened later for a calcified artery than for a normally elastic artery. Accordingly, determination of inflation and deflation of the pressure devices 12 is preferably adjusted for every individual subject to be treated. In a conventional system, both the timing of inflation and deflation as well as the pressure of the pressure pulse is adjusted manually.

There are two basic safety criteria: (1) the inflation valves must not be opened so that the pressure pulse wave reaches the root of the aorta during systole, forcing the aortic valve to close prematurely, thereby creating systolic loading; and (2) the deflation valves are opened to the atmosphere before the next R wave 504 to allow enough time for the air pressure in the pressure devices 12 to decay so there is no significant residual pressure causing a tourniquet effect.

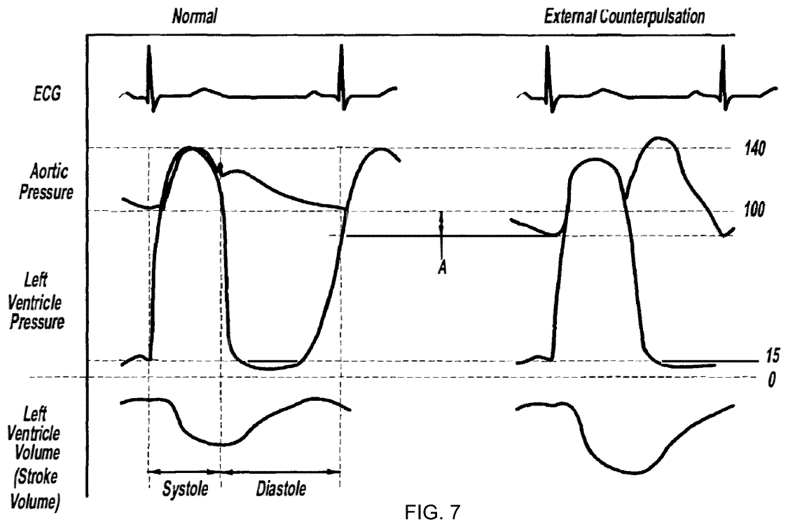

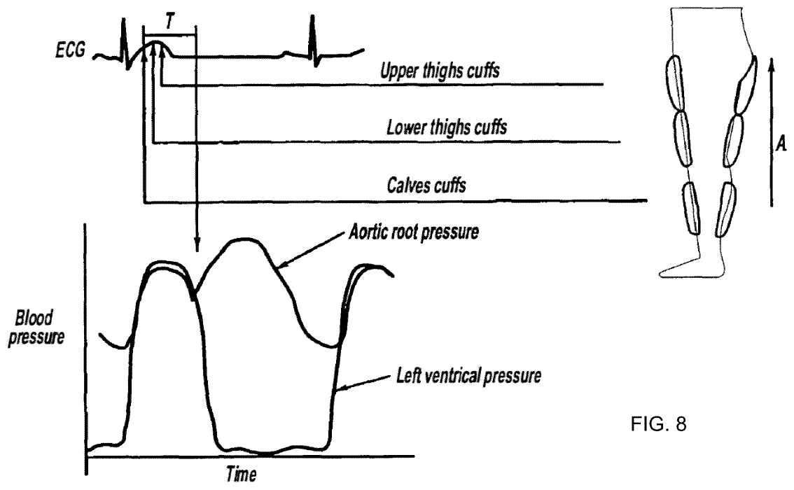

The left to side of FIG. 8 of the '702 patent, recreated here as FIG. 7 illustrates a normal graph of the aortic pressure relative to the ECG, whereas the right side illustrates the aortic pressure with appropriate timing for inflation and deflation as well as the appropriate pressure for the pulse. Again, the pressure can be adjusted manually in conventional systems, e.g., from 0-6 PSI, to achieve the waveform for the aortic pressure illustrated on the right side of FIG. 7. The timing for the start of the pressure pulse can also be manually adjusted in a conventional system. FIG. 8, which is a recreation of FIG. 7 in the '702 patent, illustrates example sequential timing of pressure devices 12, relative to the T-wave 506.

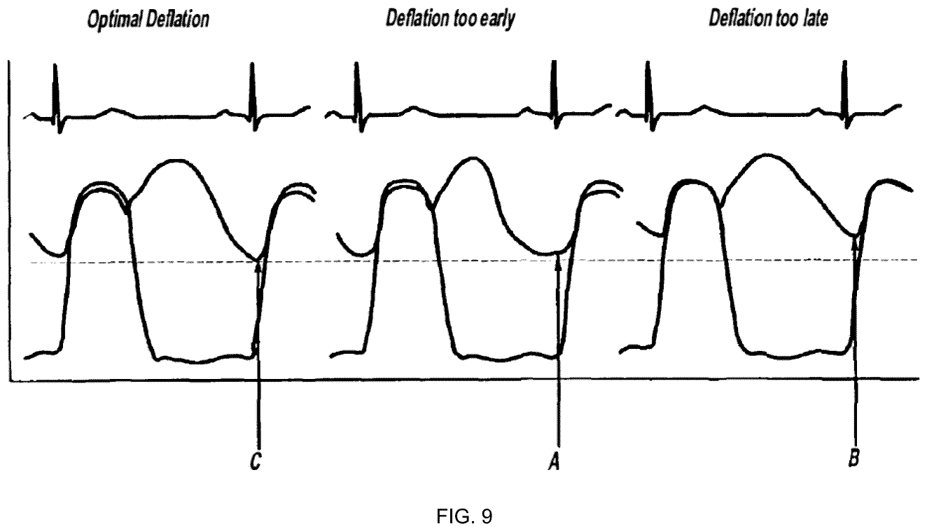

When deflation valves open too early, the end diastolic pressure is flat, as shown at A in the middle panel of FIG. 9, which is a recreation of FIG. 9 of the '702 patent. In this circumstance, blood from the upper portion of the body (i.e., head and neck) fills the peripheral vascular bed instead of the blood from the left ventricle, negating the “sucking effect” and reduction of systolic pressure by opening up the emptied vascular bed that has been previously compressed during diastole. In addition, opening the deflation valves too early reduces the area under the diastolic augmentation curve, thereby reducing coronary blood flow and energy supply to the myocardium. Further, if the deflation valves are opened too late, as shown at B in the right panel of FIG. 9, end diastolic pressure increases and the left ventricle spends more energy in isovolumetric contraction to generate more pressure before ejection begins. The left panel of FIG. 9 demonstrates timing the opening of the deflation valves to achieve maximum decrease in end diastolic (or presystolic) blood pressure, as indicated at C.

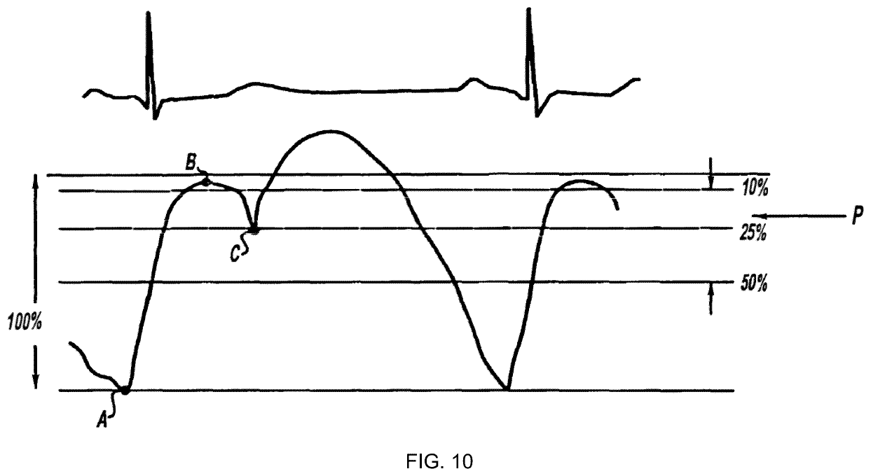

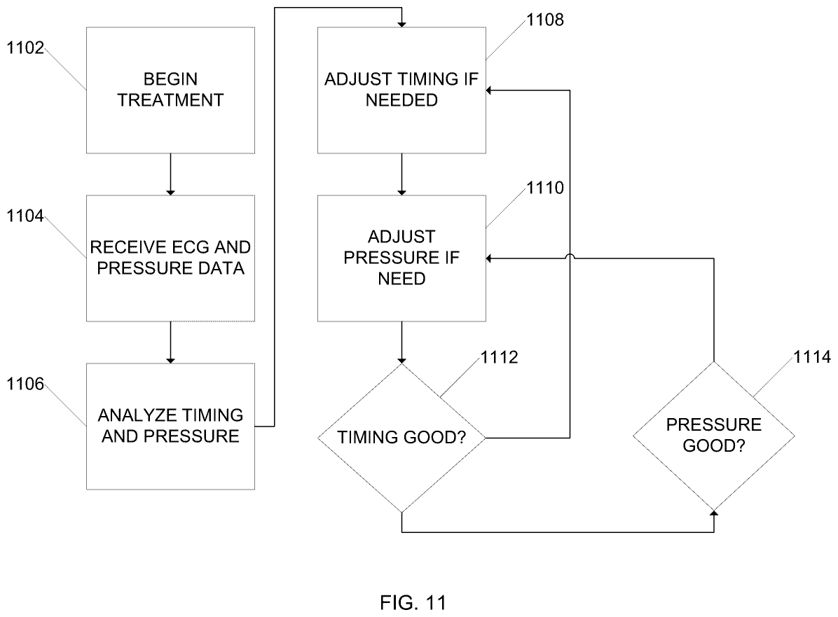

Thus, as noted, the technician can adjust the timing and pressure to achieve the waveform on the right side of FIG. 9. But, as noted in the '702 patent, there are circumstances when the exact time at which aortic valve closes is difficult to detect, such as when noninvasive detection methods are used (e.g., finger plethys-mograph) and a dicrotic notch does not appear on the resulting waveform. The dicrotic notch may not be visible because it is composed of a high-frequency wave that is attenuated much faster when transmitting through the vascular bed. In this case, as exemplified in FIG. 11 of the '702 patent, recreated her as FIG. 10, the inflation time is adjusted by approximation, such that the augmented diastolic waveform begins when the systolic pressure has dropped from about ten (10) percent to about fifty (50) percent of the distance from the end diastolic pressure (A) to the peak systolic pressure (B). Thus, the pressure drop from peak systolic pressure (B) to the beginning of diastolic augmentation pressure (C) is from about ten (10) percent to about fifty (50) percent, preferably about twenty-five (25) percent to about fifty (50) percent, of the difference in pressure between the end diastolic pressure (A) and peak systolic pressure (B); or B−C=x(B−A), where x is from about 0.1 to about 0.5, preferably 0.25 to 0.5. This approximation is derived from the observation that the counter pulsation pulse arrives at the root of the aorta, i.e., point C in FIG. 10, when the peak systolic pressure has dropped approximately ten (10) to fifty (50) percent, as shown at P.

As noted above, in conventional systems, a technician typically observes the treatment locally via a device 10 and then attempts to achieve the optimal timing and pressure to achieve the waveform on the right of FIG. 9, by manually adjusting the timing and pressure. But in certain embodiments of the systems and methods described herein, the information ECG and blood pressure information can be observed remotely. But regardless of whether remote or not, application 112 and/or 132 can be configured to monitor the ECG and blood pressure information and automatically adjust the pressure and timing of the inflation of pressure devices 12. In other words, application 112/132 can comprise a timing engine that analysis the ECG and pressure information and automatically adjust the inflation timing to achieve optimal timing. Application 112/132 can also comprise a pressure monitoring engine to monitor the pressure and adjust it to achieve optimal pressure.

Thus, as illustrated in the flow chart of FIG. 11, the treatment can begin in step 1102. The measured ECG and pressure data can then be received by application 112/132 in step 1104 and analyzed in step 1106. Based on that analysis, the timing and pressure can be automatically adjusted in steps 1108 and 1110. The updated timing a pressure can then be checked in steps 1112 and 1114 and if further adjustments are needed, then they can again be adjusted automatically until the optimal timing and pressure are achieved.

Of course, it is likely that the automated adjustment will need to be observed by a technician or doctor, but the remote capabilities allow for that to happen easier and for multiple patients at once. But the automated adjustment with remote monitoring can greatly improve the delivery and efficiency of treatment.

### 2.2. EMR Integration

Moreover, as noted above, there is in a conventional system an initial calibration of the pressure and timing, but then a more granular adjustment that needs to occur due to the patient specific condition and parameters. But in the systems and methods described herein, device 10 can be tied into, e.g., the Electronic Medical Record (EMR) of the patient and therefore have access to patient specific information. Thus, the initial calibration can actually be based on the patient specific information.

Moreover, data from each treatment can be automatically used to update EMR, or other data that can then be used for future treatments. Again, this will increase the effectiveness and efficiency of treatment. And ensure that the EMR data is up to date.

### 2.3. Automatic EMR Analysis

EMR integration can also help to identify patients for whom ECP treatment is appropriate. As noted above, ECP is approved for treating chronic stable angina that doesn't respond to other treatments. But the doctor has to identify patients that would benefit and be approved. But application 112/132 can comprise a patient identification engine that can analyze the EMR data and identify eligible patients.

Currently, the doctor will have to review the entire EMR to select the right patients for treatment. The, e.g., AI-empowered device 10 pre-selects the cases and presents them to the doctor for approval. This saves a great amount of time as a doctor, previously, had to take almost one hour to study each case and it would take only 5-6 minutes to review the data provided by the, e.g., device 10. It saves 90% of the doctor's time from going through paperwork. The selected patients can then be, e.g., notified by the nurse and prepared for pre-authorization by insurance. Since ECP device 10, as described herein is integrated with the EMR, this entire current manual process is no longer required. This increases the efficiency of the team and patient recruitment and preapproval is made easier and more efficient.

Once the patient selection is done and the cardiologist has recommended the treatment, the operational staff of the medical technician and the nurse oversee the daily treatment process. There are many aspects such as placement of electrodes, the pressure of the cuffs (12) and treatment details that are stored in the database 114/134 and the device 10 prompts the team timely with the right suggestions for better patient care and safety.

The most crucial aspect of treatment is getting the right waveform (see right side of FIG. 9) for increasing the blood supply to the heart. When the heart is relaxing in diastole, exactly sequential inflation must be initiated and just before the end of diastole it must be deflated. Many times, the operators are not well trained to get the nuances of the fine-tuning of the device 10. With an, e.g., AI-empowered ECP device 10, the operator can be prompted to make changes in the settings of the device 10 so the patient can get the best waveforms which increases the success rate of the treatment. Or, as noted, the adjustments can be made automatically.

As ECP device 10 has a remote cloud monitoring function, the waveforms can be viewed by the physician for accuracy. If the team is new, training will be easier and physician intervention is possible in time.

With EMR integration, the physician can manage all the necessary data and daily reporting. The medical billing team can have access to submitting to insurance. This device gives much more time to the operating team and multiple devices can be set up in various locations ensuring patient safety and successful results. Automatic billing is important. Due to the EMR integration, the patient and/or payor can be automatically billed. This increases efficiency immensely for a typical doctor's practice.

There are 12 million cases of chronic resistant angina in the US alone and the number is growing. Today treatment as described above is grossly underutilized due to all the above disadvantages making it difficult for the doctors and team as it is very time and effort-consuming compared to the reimbursement offered by the insurance.

The above description of the disclosed embodiments is provided to enable any person skilled in the art to make or use the invention. Various modifications to these embodiments will be readily apparent to those skilled in the art, and the general principles described herein can be applied to other embodiments without departing from the spirit or scope of the invention. Thus, it is to be understood that the description and drawings presented herein represent a presently preferred embodiment of the invention and are therefore representative of the subject matter which is broadly contemplated by the present invention. It is further understood that the scope of the present invention fully encompasses other embodiments that may become obvious to those skilled in the art and that the scope of the present invention is accordingly not limited.

As used herein, the terms “comprising,” “comprise,” and “comprises” are open-ended. For instance, “A comprises B” means that A may include either: (i) only B; or (ii) B in combination with one or a plurality, and potentially any number, of other components. In contrast, the terms “consisting of,” “consist of,” and “consists of” are closed-ended. For instance, “A consists of B” means that A only includes B with no other component in the same context.

Combinations, described herein, such as “at least one of A, B, or C,” “one or more of A, B, or C,” “at least one of A, B, and C,” “one or more of A, B, and C,” and “A, B, C, or any combination thereof” include any combination of A, B, and/or C, and may include multiples of A, multiples of B, or multiples of C. Specifically, combinations such as “at least one of A, B, or C,” “one or more of A, B, or C,” “at least one of A, B, and C,” “one or more of A, B, and C,” and “A, B, C, or any combination thereof” may be A only, B only, C only, A and B, A and C, B and C, or A and B and C, and any such combination may contain one or more members of its constituents A, B, and/or C. For example, a combination of A and B may comprise one A and multiple B's, multiple A's and one B, or multiple A's and multiple B's.

What is claimed is:

1. A system comprising:
at least one pressure device configured to inflate and deflate;
at least one hardware processor; and
software that is configured to, when executed by the at least one hardware processor,
automatically initiate inflation and deflation of the at least one pressure devices as part of a treatment,
receive ECG and pressure data,
analyze the ECG and pressure data and determine timing and pressure required to achieve inflation and deflation of the at least one pressure device in synchrony with the subject's cardiac cycle, and
automatically adjust at least one of timing and pressure of the inflation and deflation of the at least one pressure device to achieve synchrony with the subject's cardiac cycle.

2. The system of claim 1, wherein inflation and deflation are affected so as to create a retrograde pulse of arterial blood that arrives at the heart at approximately the end of the ejection phase of the left ventricle at the time of aortic valve closure, and wherein the inflation volume and timing is automatically adjusted to achieve synchrony with the subject's cardiac cycle.

3. The system of claim 1, wherein the software is further configured to, when executed by the at least one hardware processor, receive information related to the subject's specific medical conditions and initiate inflation and deflation of the at least one pressure devices based on the subject specific information.

4. The system of claim 1, wherein the software is further configured to, when executed by the at least one hardware processor, monitor the ECG and pressure data and continually adjust the timing and or pressure to achieve inflation and deflation of the at least one pressure device in synchrony with the subject's cardiac cycle.

5. The system of claim 1, wherein the at least one hardware processor is remote from the at least one pressure device.

6. The system of claim 3, wherein the software is further configured to, when executed by the at least one hardware processor, to receive information related to the treatment and update the information related to the subject's specific medical conditions.

7. A method comprising:
automatically initiating inflation and deflation of the at least one pressure devices as part of a treatment,
receiving ECG and pressure data,
analyzing the ECG and pressure data and determine timing and pressure required to achieve inflation and deflation of the at least one pressure device in synchrony with the subject's cardiac cycle, and
automatically adjusting at least one of timing and pressure of the inflation and deflation of the at least one pressure device to achieve synchrony with the subject's cardiac cycle.

8. The method of claim 6, wherein inflation and deflation are affected so as to create a retrograde pulse of arterial blood that arrives at the heart at approximately the end of the ejection phase of the left ventricle at the time of aortic valve closure, and wherein the inflation volume and timing is automatically adjusted to achieve synchrony with the subject's cardiac cycle.

9. The system of claim 6, further comprising receiving information related to the subject's specific medical conditions and initiate inflation and deflation of the at least one pressure devices based on the subject specific information.

10. The system of claim 6, further comprising monitoring the ECG and pressure data and continually adjust the timing and or pressure to achieve inflation and deflation of the at least one pressure device in synchrony with the subject's cardiac cycle.

11. The system of claim 9, further comprising receiving information related to the treatment and update the information related to the subject's specific medical conditions.
